(19) United States
(12) Patent Application Publication
(10) Pub. No.: US 2026/0133182 A1
(43) Pub. Date: May 14, 2026

(54) METHOD OF DETERMINING FUSOGENIC POTENTIAL OF SPERM AND USE THEREOF IN ASSISTED FERTILIZATION AND DRUG SCREENING

(71) Applicant: Technion Research & Development Foundation Limited, Haifa (IL)

(72) Inventors: Benjamin PODBILEWICZ, Haifa (IL); Nicolas Gaston BRUKMAN, Haifa (IL); Clari VALANSI, Haifa (IL)

(73) Assignee: Technion Research & Development Foundation Limited, Haifa (IL)

(21) Appl. No.: 19/390,570

(22) Filed: Nov. 16, 2025

Related U.S. Application Data
(63) Continuation of application No. PCT/IL2024/050485, filed on May 16, 2024.
(60) Provisional application No. 63/466,748, filed on May 16, 2023.
(60) Provisional application No. 63/622,112, filed on Jan. 18, 2024.

(51) Int. Cl. G01N 33/50

(52) U.S. Cl. CPC G01N 33/5091

(57) ABSTRACT

A method of determining fusogenic potential of sperm is provided. The method comprising: (a) contacting a biological sample comprising sperm with somatic cells expressing ectopic JUNO under conditions which allow binding of the sperm to the JUNO; and subsequently (b) detecting multinucleation in the somatic cells, wherein a level of the multinucleation is indicative of the fusogenic potential of the sperm.

### RELATED APPLICATIONS

This application is a Continuation of PCT Patent Application No. PCT/IL2024/050485 having International filing date of May 16, 2024 which claims the benefit of priority under 35 USC § 119(e) of U.S. Provisional Patent Application No. 63/622,112 filed on Jan. 18, 2024 and U.S. Provisional Patent Application No. 63/466,748 filed on May 16, 2023. The contents of the above applications are all incorporated by reference as if fully set forth herein in their entirety.

### FIELD AND BACKGROUND OF THE INVENTION

The present invention, in some embodiments thereof, relates to a method of determining fusogenic potential of sperm and use thereof in assisted fertilization and drug screening.

Infertility is estimated to affect approximately 15% of the population (World Health Organization 2023), and assisted Reproductive techniques (ARTs) represent a powerful tool for assisting couples that deal with this burden. Among them, the in vitro fertilization (IVF) (Steptoe and Edwards 1978), in which retrieved oocytes are inseminated under laboratory conditions, overcomes many defects that are responsible for defective fertility. Since 1992, it is also possible to inject a single sperm into an oocyte using a microinjector with the aim to by-pass all natural barriers of fertilization (Palermo et al. 1992). The latter is called intracytoplasmic sperm injection (ICSI) and despite its increasing usage, the routine usage of this technique is under debate as it was associated with a slightly higher risk of adverse outcomes in the progeny (Practice Committees of the American Society for Reproductive Medicine and the Society for Assisted Reproductive Technology 2020).

During the fertility evaluation of a male patient, a basic semen examination is performed where different sperm parameters are determined: concentration, motility, morphology and vitality (World Health Organization, HRP 2021). This can be complemented with additional tests to determine the fertilizing potential of the sperm and choose the best treatment for the couple. The hamster oocyte penetration (HOP) test has been proposed as a quantitative method for analyzing the fusogenic potential of human spermatozoa (Aitken and Elton 1986), however, it was excluded from the newest WHO laboratory manual for the examination and processing of human semen (World Health Organization, HRP 2021) for being considered obsolete. Therefore, to date there is no other standardized methodology to analyze the ability of human sperm to fuse to human oocytes.

In mammals, the adhesion of the sperm and the oocyte plasma membranes is mediated by the species-specific interaction of two molecules: IZUMO1 and JUNO (Bianchi and Wright 2015 ). The transmembrane protein IZUMO1 is expressed during spermatogenesis and localizes to the fusogenic region of the sperm head after an exocytic process named acrosome reaction (Naokazu Inoue et al. 2005; Satouh et al. 2012). On the other hand, the IZUMO1's receptor, JUNO, is an oocyte-specific protein bound to the plasma membrane by a GPI anchor (Bianchi et al. 2014). The subsequent fusion of the two gametes relies on the action of IZUMO1 in a unilateral manner (Brukman et al. 2023). The IZUMO1-JUNO interaction is conserved also in humans (Aydin et al. 2016; Ohto et al. 2016) and its relevance for human fertilization is supported by data obtained from infertile patients (Clark and Naz 2013; Yu et al. 2018; Enoiu et al. 2022).

Recently, it was found that mouse sperm can fuse to fibroblasts ectopically expressing the sperm receptor JUNO (Brukman et al. 2023). No syncytia was reported.

### SUMMARY OF THE INVENTION

According to an aspect of some embodiments of the present invention there is provided a method of determining fusogenic potential of sperm, the method comprising:
(a) contacting a biological sample comprising sperm with somatic cells expressing ectopic JUNO under conditions which allow binding of the sperm to the JUNO; and subsequently
(b) detecting multinucleation in the somatic cells, wherein a level of the multinucleation is indicative of the fusogenic potential of the sperm.

According to some embodiments of the invention, the cells comprise epithelial cells.

According to some embodiments of the invention, the cells comprise fibroblasts.

According to some embodiments of the invention, the biological sample is a human sample.

According to some embodiments of the invention, the cells are human cells.

According to some embodiments of the invention, the cells are non-human cells.

According to some embodiments of the invention, the cells express at least one detectable marker for the multinucleation.

According to some embodiments of the invention, the cells comprise at least 2 subpopulation of cells each expressing a distinct detectable marker for the multinucleation.

According to some embodiments of the invention, the detectable marker for the multinucleation is fluorescent or chromogenic.

According to some embodiments of the invention, the detectable marker for the multinucleation is nuclear.

According to some embodiments of the invention, the detecting comprise fluorescence or chromogenic detection.

According to some embodiments of the invention, the detecting is by a nuclear stain or a nuclear localized reporter polypeptide.

According to some embodiments of the invention, the conditions support capacitation of the sperm.

According to some embodiments of the invention, the conditions comprise albumin and Ca⁺².

According to some embodiments of the invention, when the level is above a predetermined threshold, it is indicative of competent sperm.

According to some embodiments of the invention, when the level is below a predetermined threshold, it is indicative of defective sperm.

According to some embodiments of the invention, the level is calculated as % of multinucleation=[multinucleated cells (NuM)/total number of nuclei fluorescent cells (NuF)]×100.

According to some embodiments of the invention, the sperm is naïve.

According to an aspect of some embodiments of the present invention there is provided a method of assisted fertilization in a subject in need thereof, the method comprising:
qualifying sperm for use in the assisted fertilization according to the method as described herein;
wherein when the sperm is found competent, directing the subject to intrauterine insemination (IUI) or in vitro fertilization (IVF) and wherein when the sperm is found defective directing the subject to intracytoplasmic sperm injection (ICSI).

According to an aspect of some embodiments of the present invention there is provided a method of identifying an agent which affects fusogenic potential of sperm, the method comprising:
(a) contacting an aliquoted biological sample comprising sperm and somatic cells expressing ectopic JUNO under conditions which allow binding of the sperm to the JUNO in presence and in absence of an agent; and subsequently
(b) detecting multinucleation in the somatic cells in the presence and in the absence of the agent, wherein a change in level of the multinucleation is indicative of the effect of the agent on the fusogenic potential of the sperm.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWING(S)

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings. Specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a summary of a sequence of steps of the assay of some embodiments of the invention;

FIGS. 2A-D shows sperm induce syncytia formation of fibroblasts. (A) Scheme of experimental design: Baby Hamster Kidney (BHK) cells were transfected with either pCI::H2B-RFP or pCI::JUNO::H2B-RFP vectors and pcDNA3.1-EGFP-MBD-nls. Mouse sperm were obtained from the epididymis of adult mice and capacitated in HTF capacitating medium. Sperm cells were co-incubated with the BHK cells for 4 h and then the cells were fixed and stained with DAPI to detect the DNA. (B) Representative images showing H2B-RFP (magenta), GFP-MBD (green), DAPI (blue) channels and the merge. Dotted lines contour relevant cells. Arrowheads point to fused sperm (GFP positive). The presence of cycloheximide is indicated (CXH). Scale bars, 20 μm. (C) The percentage of multinucleation was defined as the ratio between the nuclei in multinucleated cells (NuM) and the total number of nuclei fluorescent cells (NuF), as follows: % of multinucleation=(NuM/NuF)×100. Individual data is shown and means±SEM of six independent experiments. The number of nuclei counted per experiment and per treatment was 500. For JUNO transfected cells, multinucleation was counted separately for cells with and without sperm fused to them. Comparisons were made with one-way ANOVA followed by Tukey's test. ns=non-significant, ****P<0.0001. (D) In another set of experiments JUNO-transfected cells were treated with 200 μg/ml CXH to inhibit de novo synthesis of proteins. Shown individual data and means±SEM of five independent experiments. ns=non-significant, **P<0.01.

FIGS. 3A-C show IZUMO1 diffuses into the target cell upon sperm fusion. (A) BHK were transfected with pcDNA3.1-EGFP-MBD-nls alone or together with pCI::JUNO::H2B-RFP and co-incubated with sperm obtained from transgenic mice expressing IZUMO1-mcherry. Representative images showing H2B-RFP and Izumo1-mCherry (magenta), GFP-MBD (green), DAPI (blue) channels and the merge. Dotted lines contour relevant cells. Scale bar, 20 μm. Magenta arrowheads point to unfused sperm (GFP negative) presenting IZUMO1-mCherry signal while green arrowheads point to fused sperm (GFP positive) without Izumo1-mCherry signal. (B) BHK were transfected with pcDNA3.1-EGFP-MBD-nls together with pCI::JUNO::H2B-RFP and co-incubated with wild-type mouse sperm. After fixation an immunostaining against IZUMO was performed. Representative images showing H2B-RFP (magenta), GFP-MBD (green), IZUMO1 staining (yellow), DAPI (blue) channels and the merge. Scale bar, 20 μm. Yellow arrows point to unfused sperm (GFP negative) presenting IZUMO1 signal while green arrowheads point to fused sperm (GFP positive) without IZUMO1 signal. (C) Models for sperm-induce cell-cell fusion. In general, this phenomenon resembles the so-called “fusion from without” in which viruses can induce the fusion of the target cells (Falke, Knoblich, and Müller 1985; Tang et al. 2021; Edwards and Brown 1986). i—One sperm fuses with a BHK, IZUMO1 is transferred by diffusion and mediates the fusion to another cell. ii—One sperm fuses simultaneously to two BHK bridging them and inducing the formation of a syncytium.

FIGS. 4A-C show sperm-induced fusion is dependent on JUNO. (A) Scheme of experimental design. BHK cells transfected with pCI::GFPnes or pCI::H2B-RFP empty vectors or containing the coding sequence for the expression of JUNO were mixed as indicated. Later, the cells were co-incubated with sperm for 4 h, fixed and stained with DAPI. (B) Representative images for each treatment. Each separate channel for GFPnes (green cytoplasm), H2B-RFP (magenta nuclei) and DAPI staining (blue) are shown. Scale bars, 20 μm. Fused, mixed cells mixed cells containing both GFPnes and H2B-RFP staining. (C) Quantification of content mixing experiments. The percentage of mixing was defined as the ratio between the nuclei in mixed cells (NuM) and the total number of nuclei in mixed cells and fluorescent cells in contact that did not fuse (NuC), as follows: % of mixing=(NuM/[NuM +NuC])×100. Bar chart showing individual experiment values (each corresponding to 1,000 nuclei) and means±SEM of six independent experiments. Comparisons by one-way ANOVA followed by Tukey's test. ns=non-significant, ***P<0.001, ****P<0.0001.

FIGS. 5A-B show syncytia formation requires a functional sperm, is dependent on IZUMO1 and is not affected by an inhibitor of cell division. BHK cells were transfected with pcDNA3.1-EGFP-MBD-nls together with pCI::H2B-RFP (empty vector) or pCI::JUNO::H2B-RFP and co-incubated with control sperm in the presence of 20 μM of the inhibitor of cell division FdUdr or of 1 μg/μl of anti-IZUMO1 antibody (clone Mab120). Alternatively, cells were incubated with sperm incubated in a medium lacking calcium (—Ca²⁺) or bovine serum albumin (—BSA), none of them supporting capacitation. The number of sperm fused per 500 fluorescent cells (A) and the percentage of multinucleation (B) were determined. Bar charts showing individual experiment values and means±SEM of five independent experiments. Comparisons by one-way ANOVA followed by Tukey's test. In red are the comparisons against the empty vector. ns=non-significant, ***P<0.001, ****P<0.0001.

FIGS. 6A-C show sperm induced BHK-BHK fusion in a species specific manner. BHK cells were transfected with pcDNA3.1-EGFP-MBD-nls together pCI::H2B-RFP (empty vector) or pCI::JUNO::H2B-RFP encoding for human JUNO (hJUNO) or mouse JUNO (mJUNO), and co-incubated with capacitated mouse sperm (A) Representative images showing H2B-RFP (magenta), GFP-MBD (green), DAPI (blue) channels and the merge. Dotted lines contour a cell transfected with human JUNO with two mouse sperm fused (arrowheads). Scale bar, 20 μm. The number of sperm fused per 500 fluorescent cells (B) and the percentage of multinucleation (C) were determined. Bar charts showing individual experiment values and means±SEM of five independent experiments. Comparisons by one-way ANOVA followed by Tukey's test. In red are the comparisons against the empty vector. *P<0.05, **P<0.01, ***P<0.001;

FIG. 7 is a schematic illustration showing a specific embodiment of the assay, employing cells in culture ectopically expressing the egg protein JUNO and a DNA green fluorescent marker. Upon the incubation with fertilizing competent sperm, the fusion of the cells is induced and multinucleated cells are produced. This is taken as a readout to determine whether the sperm is suitable for different assisted reproductive techniques: intrauterine insemination (IUI), conventional in vitro fertilization (IVF) or intracytoplasmic sperm injection (ICSI).

FIGS. 8A-B shows the amount of sperm cells required to induce multinucleation. Baby hamster kidney (BHK) cells in 24-well plates were transfected with pCI::JUNO::H2B-RFP vectors and pcDNA 3.1-EGFP-MBD-nls co-incubated with capacitated sperm for 4 hr. (A) The distribution of BHK cells with 1, 2, 3, or 4 nuclei presenting different amounts of sperm fused to them. Bigger syncytia tend to contain more sperm. (B) Different amounts of sperm were added to the cells and the percentage of multinucleation was determined. To each well, 1.5-3×10⁶ can produce statistically-significant increase in the percentage of multinucleation compared to the control without sperm, indicating that the levels of multinucleation were dependent on the amount of sperm added to the cells (***p<0.001, ****p<0.0001).

FIG. 9 shows that cell-cell fusion correlates with the fertilizing ability of sperm during in vitro fertilization assays. Multinucleation levels are relative to the control without sperm as a function of the percentage of fertilized eggs when cumulus-oocytes complexes were used (Left panel), or as a function of the number of sperm fused per oocytes when ZP-free eggs were employed (Right panel). Each dot corresponds to a different mouse. The Pearson's coefficient ‘r’ and the significance are included in each panel.

FIG. 10A-B show that the acrosome reaction is reduced in non-capacitated sperm (control for FIG. 5A-B), but does not correlate with sperm-induced cell-cell fusion requiring JUNO (SPICER) levels in capacitated conditions. (A) Percentage of acrosome reaction of fresh sperm, capacitated sperm, and sperm incubated in media lacking calcium (—Ca²+) or bovine serum albumin (-BSA). Comparisons by one-way ANOVA followed by Tukey's test. ns=non-significant, **p<0.01, ****p<0.0001. (B) Multinucleation levels relative to the control without sperm as a function of the percentage of acrosome reaction after capacitation. BHK multinucleation did not correlate with the percentage of acrosome reaction of capacitated sperm, suggesting that the process relies not only on capacitation but on the overall sperm fertilizing potential. Each dot corresponds to a different mouse. The Pearson's coefficient ‘r’ and the significance are included.

FIGS. 11A-C show that the timing of sperm fusion to somatic cells is critical for content mixing. (A) Scheme of experimental design. Baby hamster kidney (BHK) cells transfected with pCI::JUNO::GFPnes were incubated or not with sperm for 1 hr. When indicated, the sperm were washed and cells previously transfected with pCI::H2B-RFP, pCI::JUNO::H2B-RFP, or pCI::VSV-G::H2B-RFP were added. After 18 hr, the cells were fixed, and stained with DAPI. To activate VSV-G, a treatment with low pH was done. In parallel, the regular mixing assay was performed in which BHK cells transfected with pCI::JUNO::GFPnes or pCI::JUNO::H2B-RFP were previously mixed and then co-incubated (or not) with sperm for 4 hr, fixed, and stained with DAPI. (B) Quantification of content mixing experiments. The percentage of mixing was defined as the ratio between the nuclei in mixed cells (NuM) and the total number of nuclei in mixed cells and fluorescent cells whose cell bodies are in contact that did not fuse (NuC), as follows: % of mixing=(NuM/[NuM +NuC])×100. A bar chart showing individual experiment values (each corresponding to 1000 nuclei) and means±SEM of four independent experiments. For the 18 hr mixing: comparisons by one-way ANOVA followed by Dunnett's test against the negative control. For the 4 hr mixing: comparison by t-Student's test. ns=non-significant, ***p<0.001, ***p<0.0001. (C) Multinucleation was quantified for the pCI::JUNO::GFPnes-transfected cells for the indicated conditions. Individual data is shown and means±SEM of four independent experiments. Comparisons by one-way ANOVA followed by Tukey's test. ns=non-significant, ***p<0.0001.

### DESCRIPTION OF SPECIFIC EMBODIMENTS OF THE INVENTION

The present invention, in some embodiments thereof, relates to a method of determining fusogenic potential of sperm and use thereof in assisted fertilization and drug screening.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

During the fertility evaluation of a male patient, a basic semen examination is performed where different sperm parameters are determined: concentration, motility, morphology and vitality (World Health Organization, HRP 2021). This can be complemented with additional tests to determine the fertilizing potential of the sperm and choose the best treatment, in case needed. To date there is no other standardized methodology to analyze the ability of sperm to fuse to oocytes.

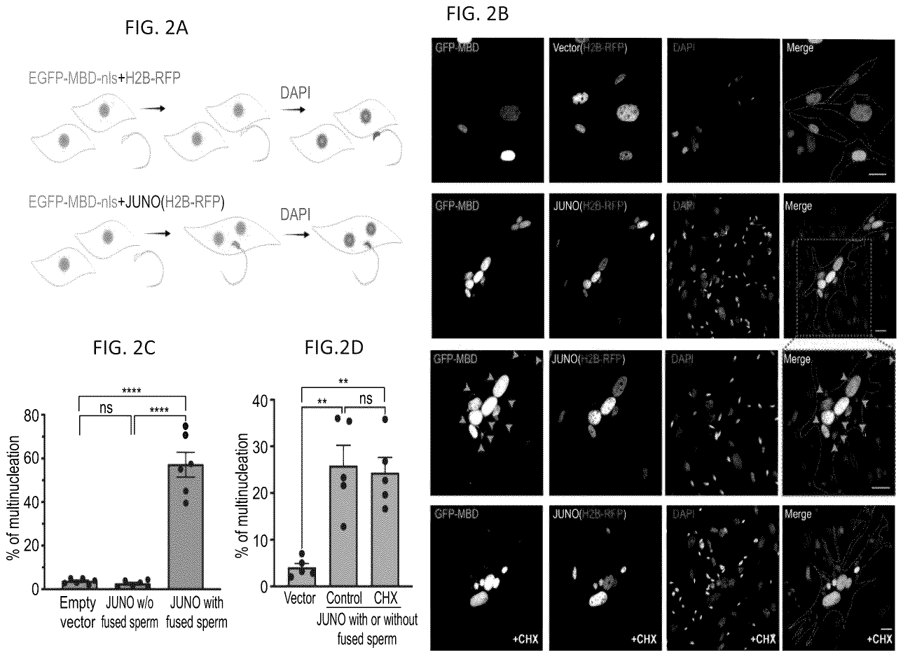

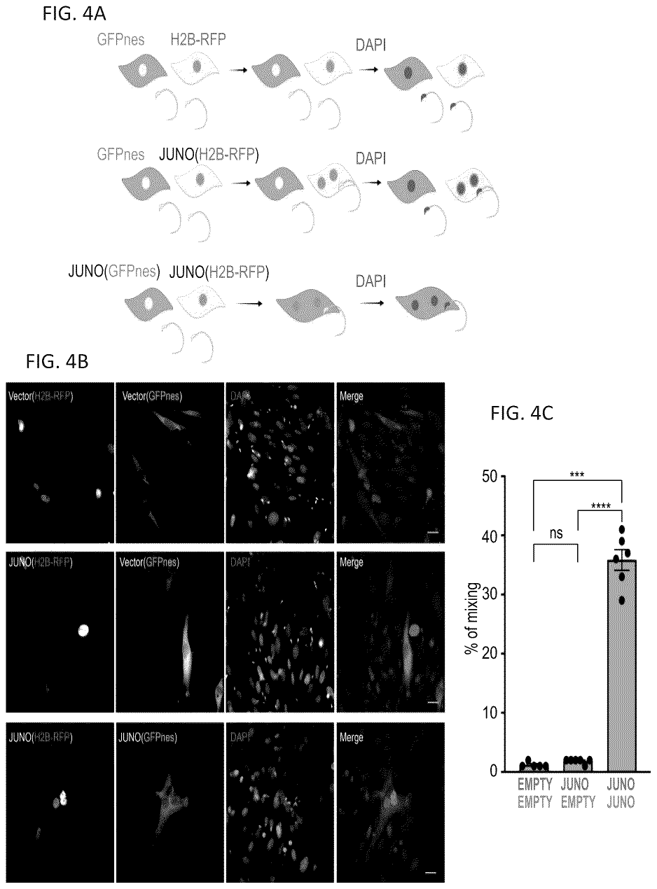

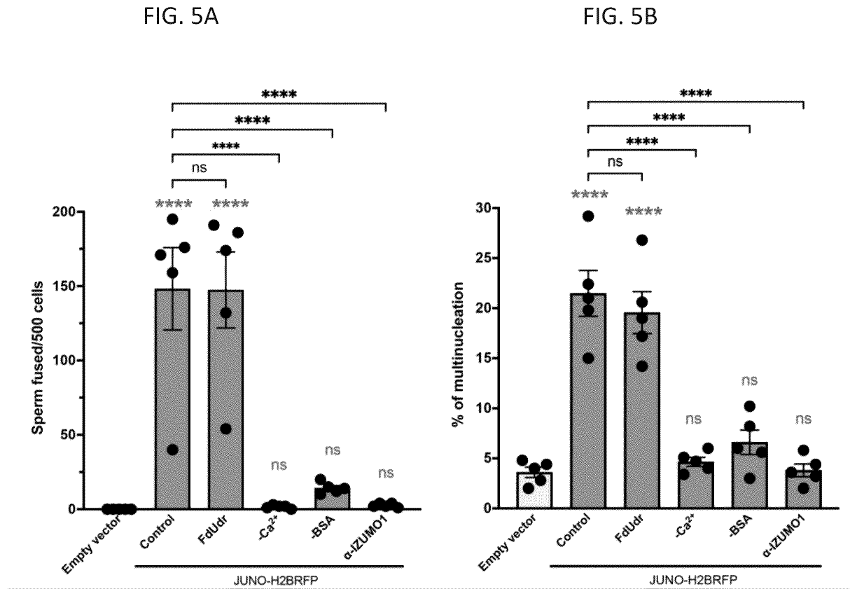

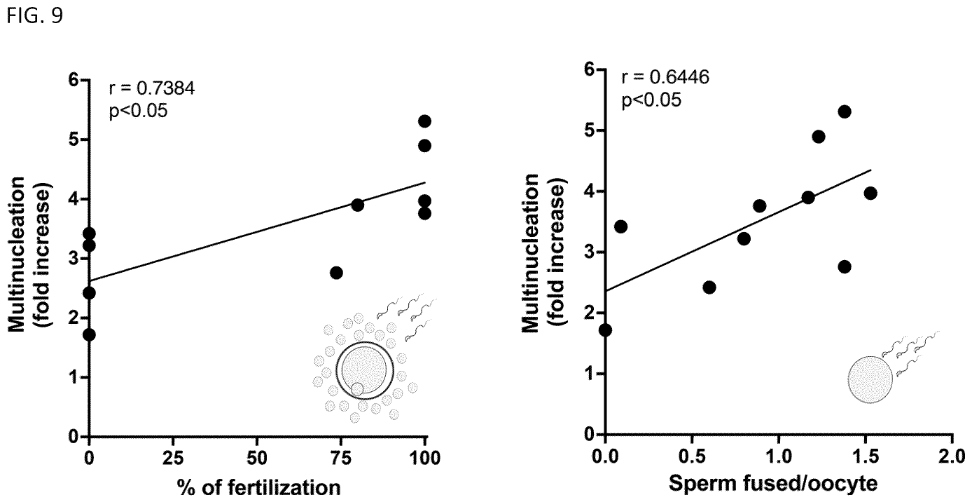

Whilst conceiving and reducing to practice embodiments of the invention the present inventors developed a novel assay for assessing the fusogenic potential of sperm. The assay is based on the species-specific ability of sperm cells which normally express IZUMO1 to bind somatic cells genetically modified to express its receptor, JUNO, and fuse thereto resulting in multinucleation (FIGS. 2A-D). The present inventors have shown that the extent of cell-cell fusion. as assayed by the level of multinucleation (syncytia formation), correlates with the sperm fertilizing ability (FIG. 9). The fusion is JUNO-dependent as shown in FIGS. 4A-C. Through the use of a monoclonal anti IZUMO1 antibody; the present inventors have shown that fusion is mediated through the binding of IZUMO1 to its receptor. In addition, the assay is dependent on sperm capacitation (FIGS. 5A-B) and the overall sperm fertilizing ability (FIG. 9). These results support the use of this assay as a predictor of sperm fertilizing ability.

Thus, according to an aspect of the invention there is provided a method of determining fusogenic potential of sperm, the method comprising:
(a) contacting a biological sample comprising sperm with somatic cells expressing ectopic JUNO under conditions which allow binding of said sperm to said JUNO; and subsequently
(b) detecting multinucleation in said somatic cells, wherein a level of said multinucleation is indicative of the fusogenic potential of the sperm.

As used herein, “a fusogenic potential” is the ability of sperm to bind tightly to the surface of the oocyte and fuse their membranes concluding the fertilization, allowing the genetic exchange and forming a single cell called zygote (See Brukman, N. G., Uygur, B., Podbilewicz, B., and Chernomordik, L. V. (2019). How Cells Fuse. J. Cel Biol. 218, 1436-1451. doi:10.1083/jcb.201901.). This fusogenic potential can be assayed by the ability of sperm to induce somatic cell-cell fusion when JUNO is ectopically expressed.

According to a specific embodiment, the sperm is a mammalian sperm.

According to a specific embodiment, the sperm is of a human being.

According to a specific embodiment, the sperm is non-human (e.g., mouse).

According to a specific embodiment, the sperm is of a ruminant, e.g., bull, sheep, goat, deer, buffalo.

According to a specific embodiment, the sperm is of a bovine.

According to a specific embodiment, the sperm is of a horse.

According to a specific embodiment, the sperm is of a pig.

According to a specific embodiment, the sperm is of a cat or any feline.

According to a specific embodiment, the sperm is of a dog or other pets. According to a specific embodiment, the sperm is of an animal in danger of extinction.

According to a specific embodiment, the sperm is of an inbreed.

According to a specific embodiment, the sperm is of a stud.

According to a specific embodiment, the sperm is of a healthy subject, such as a sperm donor.

According to a specific embodiment, the sperm is of a non-healthy subject, e.g., undergoing anti-cancer treatment such as chemotherapy or radiotherapy.

As used herein “|biological sample” refers to a biopsy or semen sample which comprises sperm.

The semen sample may have been ex-vivo analyzed or treated such as by sperm washing, semen processing, sperm capacitation, sperm cryopreservation.

According to a specific embodiment, the biological sample is assayed freshly or following sperm cryopreservation.

As used herein “somatic cells” refer to non-gametes. Such somatic cells can be of any lineage. The skilled artisan would know which cells to select, according to the assay's setting, as well as according to their culturing conditions and availability.

As mentioned, the method (assay for testing fusogenic potential) is species specific. Hence the species of the somatic cells will match that of the sperm (as part of the experimental settings).

It will be appreciated that JUNO expression is specific to the oocyte. However, when evident, expression can be endogenous or enhanced by genetic manipulation as described herein.

According to a specific embodiment, the somatic cell is a primary cell.

According to a specific embodiment, the somatic cell is a cell line.

According to a specific embodiment, the somatic cells are selected from the group consisting of epithelial cells, fibroblasts, myocytes, hepatocytes, adipocytes, immune cells, neurons, endocrine cells, blood cells, endothelial cells, osteoblasts, osteoclasts, intestinal cells, chondrocytes, glia cells, pancreatic cells, etc.

According to a specific embodiment, the somatic cells comprise epithelial cells (e.g., as HEK293, HeLa, MCF7, MDCK).

According to a specific embodiment, the somatic cells comprise fibroblasts (e.g., BHK-21, MEF, NIH/3T3).

The somatic cells express ectopic JUNO.

As used herein “JUNO” also known as “IZUMO1R” or “IZUMO1 receptor” is the receptor for IZUMO1 present at the cell surface of oocytes (oolemma), which is essential for species-specific gamete recognition and fertilization. The IZUMO1: IZUMO1R/JUNO interaction is a necessary adhesion event between sperm and egg that is required for fertilization.

The mouse ortholog was previously known as Folr4 gene (accession number: ENSMUSG00000031933). Non-limiting examples of mammalian orthologs are referred to as FOLR4, JUNO or IZUMO1R [e.g. accession numbers: ENSG00000183560 (human), XM_024975624.1 (bovine) ENSOARG00000001898 (ovine), ENSECAG00000007254 (equine), ENSSSCG00000014952 (porcine)].

The present teachings also contemplate synthetic homologs as long as the binding to the sperm's IZUMO1 is sufficient to elicit fusion and multinucleation.

Thus, according to a specific embodiment, homologous sequences of at least 60 %, 80 %, 85%, 90 %, 95 % or more, say at least 99 % identity are contemplated herein. The level of identity can be determined using dedicated software, such as BlastN/BlastP/Blast or any other global homology tool. The structural homology can be predicted using AlphaFold. The skilled artisan would know how to predict similar function, such as by assaying binding to IZUMO1.

The binding of JUNO to IZUMO1 can be qualified using any protein interaction analysis tool, such as using a reporter marker, any hybrid system, co-immunoprecipitation and the like. AlphaFold multimer can be used to predict the structural interactions between IZUMO1 and JUNO.

The somatic cells may be further genetically modified to express at least one detectable marker for somatic multinucleation.

As used herein “detectable marker” refers to any cellular, nuclear or non-nuclear that can be used to visualize nuclei.

According to a specific embodiment, the detectable marker for the multinucleation (i.e., to detect the nuclei) is fluorescent or chromogenic.

According to some embodiments, the detectable marker is a nuclear marker. Such markers can bind to DNA, or an epigenetic moiety, or a protein which is present in or on the nucleus.

According to some embodiments, a portion of the detectable maker is a protein with a nuclear localization signal (NLS), or fused to a DNA-binding domain such as the methyl-binding domain (MBD) and another portion is a fluorescent or chromogenic marker.

According to a specific embodiment, the detectable marker fused to the histoneH2B or with a nuclear localization signal.

According to an additional or an alternative embodiment, the detectable marker is expressed anywhere but the nucleus (e.g. in the cytoplasm), hence its absence marks the nucleus (dark area in the image).

For example, a reporter protein (e.g., GFP) which is attached to a nuclear export signal (NES) (e.g., see sequence of Fukuda et al. 1996 Cell. Biol. Metabol. 271(33): P 20024-28). Detectable markers that target cytoplasmic organelles like the endoplasmic reticulum, Golgi apparatus, lysosomes, endosomes or the mitochondria can be used to mark organelles which are not the nucleus.

Multinucleation which marks fusion between at least 2 cells, can also be done using at least 2 subpopulation of cells each expressing a distinct detectable marker for the multinucleation.

Thus, one population can be marked with a first fluorescent protein (e.g., GFP) and another with a second fluorescent protein (e.g., RFP). According to an embodiment of the invention, at least one of the at least 2 detectable markers is nuclear (e.g., H2B-RFP); or the 2 detectable markers are nuclear but distinctive (e.g., H2B-RFP and H2B-GFP). See for instance FIG. 4B.

It will be appreciated that the marker can, but not necessarily, stain also the sperm's nucleus following fusion. If such is desirable, the skilled artisan would take measures to elect a staining which is loose enough to bind both the somatic nucleus (by way of DNA binding) and the sperm's nucleus (e.g., EGFP-MBD, as shown in the Examples section).

According to another embodiment, in order to test cell fusion, the somatic cells comprise 2 populations of somatic cells each ectopically expressing (in addition to JUNO) a member of a dual split protein (DSP), such that fluorescent is restored only upon fusion. Examples of DSPs which can be used according to the present teachings are provided below. See for example, Nakane, Shuhei, and Zene Matsuda. “Dual Split Protein (DSP) Assay to Monitor Cell-Cell Membrane Fusion.” Methods in molecular biology (Clifton, N.J.) vol. 1313 (2015): 229-36. doi:10.1007/978-1-4939-2703-6_17///Kondo N, Marin M, Kim J H, Desai T M, Melikyan G B. Distinct requirements for HIV-cell fusion and HIV-mediated cell-cell fusion. J Biol Chem. 2015 Mar. 6; 290(10): 6558-73. doi: 10.1074/jbc.M114.623181.

Non-limiting examples of nuclear markers are provided below.

DNA Markers:
PCR Primers: Short DNA sequences used to initiate PCR amplification of specific target sequences.

DNA Probes: Single-stranded DNA molecules labeled with a detectable tag (e.g., fluorescent dye) used for hybridization-based techniques such as Southern blotting.

Methylated DNA Markers: DNA sequences marked by methyl groups, used in epigenetic studies and DNA methylation analysis.

Histone Markers:
Histone Antibodies: Antibodies specific to various histone proteins (e.g., H3, H4), used in chromatin immunoprecipitation (ChIP) assays to study histone modifications and chromatin structure.
GAPDH (Glyceraldehyde-3-phosphate dehydrogenase):
GAPDH Primers and Probes: Primers and probes specific to the GAPDH gene, often used as a reference gene in gene expression studies and quantitative PCR (qPCR) experiments for normalization purposes.

Nuclear Localization Markers:
Nuclear Staining Dyes: Fluorescent dyes (e.g., DAPI, Hoechst) that specifically bind to DNA, used for nuclear staining in fluorescence microscopy to visualize nuclei.
Nuclear Export Signals (NES): Short amino acid sequences that target proteins for export from the nucleus to the cytoplasm, used in protein engineering and subcellular localization studies.

Nuclear Pore Complex Proteins:
Nuclear Pore Antibodies: Antibodies specific to proteins of the nuclear pore complex (e.g., nucleoporins), used in immunofluorescence and immunohistochemistry to study nuclear transport and nucleocytoplasmic shuttling.

Nuclear Matrix Markers:
Lamins: Intermediate filament proteins that form the nuclear lamina, often used as markers for the nuclear matrix in cell fractionation experiments and immunofluorescence staining.

According to a specific embodiment, the somatic cells are genetically modified to ectopically express the desired proteins (e.g., JUNO and/or detectable markers) in a stable manner.

As used herein “ectopic expression” refers to a non-naturally occurring expression, such as at a level and/or genetic position which do not occur in nature. Ectopic expression may be also referred to as “heterologous expression”, “exogenous expression” or “recombinant expression”. This is usually achieved by gene insertion or genome editing, as described herein and known in the art.

To express exogenous sequences in mammalian cells, a polynucleotide sequence encoding the desired protein (e.g., JUNO and/optionally a detectable marker) is preferably ligated into a nucleic acid construct suitable for mammalian cell expression. Such a nucleic acid construct includes a promoter sequence for directing transcription of the polynucleotide sequence in the cell in a constitutive or inducible manner.

The nucleic acid construct (also referred to herein as an “expression vector”) of some embodiments of the invention includes additional sequences which render this vector suitable for replication and integration in prokaryotes, eukaryotes, or preferably both (e.g., shuttle vectors). In addition, a typical cloning vectors may also contain a transcription and translation initiation sequence, transcription and translation terminator and a polyadenylation signal. By way of example, such constructs will typically include a 5′LTR, a tRNA binding site, a packaging signal, an origin of second-strand DNA synthesis, and a 3′LTR or a portion thereof.

Preferably, the promoter utilized by the nucleic acid construct of some embodiments of the invention is active in the specific cell population transformed.

A constitutive promoter can be used although inducible ones are also contemplated herein.

Enhancer elements can stimulate transcription up to 1,000 fold from linked homologous or heterologous promoters. Enhancers are active when placed downstream or upstream from the transcription initiation site. Many enhancer elements derived from viruses have a broad host range and are active in a variety of tissues. For example, the SV40 early gene enhancer is suitable for many cell types. Other enhancer/promoter combinations that are suitable for some embodiments of the invention include those derived from polyoma virus, human or murine cytomegalovirus (CMV), the long term repeat from various retroviruses such as murine leukemia virus, murine or Rous sarcoma virus and HIV. See, Enhancers and Eukaryotic Expression, Cold Spring Harbor Press, Cold Spring Harbor, N.Y. 1983, which is incorporated herein by reference.

In the construction of the expression vector, the promoter is preferably positioned approximately the same distance from the heterologous transcription start site as it is from the transcription start site in its natural setting. As is known in the art, however, some variation in this distance can be accommodated without loss of promoter function.

Polyadenylation sequences can also be added to the expression vector in order to increase the efficiency of JUNO and/optionally a detectable marker's mRNA translation. Two distinct sequence elements are required for accurate and efficient polyadenylation: GU or U rich sequences located downstream from the polyadenylation site and a highly conserved sequence of six nucleotides, AAUAAA, located 11-30 nucleotides upstream. Termination and polyadenylation signals that are suitable for some embodiments of the invention include those derived from SV40.

In addition to the elements already described, the expression vector of some embodiments of the invention may typically contain other specialized elements intended to increase the level of expression of cloned nucleic acids or to facilitate the identification of cells that carry the recombinant DNA. For example, a number of animal viruses contain DNA sequences that promote the extra chromosomal replication of the viral genome in permissive cell types. Plasmids bearing these viral replicons are replicated episomally as long as the appropriate factors are provided by genes either carried on the plasmid or with the genome of the host cell.

The vector may or may not include a eukaryotic replicon. If a eukaryotic replicon is present, then the vector is amplifiable in eukaryotic cells using the appropriate selectable marker. If the vector does not comprise a eukaryotic replicon, no episomal amplification is possible. Instead, the recombinant DNA integrates into the genome of the engineered cell, where the promoter directs expression of the desired nucleic acid.

The expression vector of some embodiments of the invention can further include additional polynucleotide sequences that allow, for example, the translation of several proteins from a single mRNA such as an internal ribosome entry site (IRES) and sequences for genomic integration of the promoter-chimeric polypeptide. This is exemplified in the Examples section where the JUNO and H2B: RFP are separated by an IRES.

It will be appreciated that the individual elements comprised in the expression vector can be arranged in a variety of configurations. For example, enhancer elements, promoters and the like, and even the polynucleotide sequence(s) encoding JUNO and optionally the detectable marker(s) can be arranged in a “head-to-tail” configuration, may be present as an inverted complement, or in a complementary configuration, as an anti-parallel strand. While such variety of configuration is more likely to occur with non-coding elements of the expression vector, alternative configurations of the coding sequence within the expression vector are also envisioned.

Examples for mammalian expression vectors include, but are not limited to, pcDNA3, pcDNA3.1(+/−), pGL3, pZeoSV2(+/−), pSecTag2, pDisplay, pEF/myc/cyto, pCMV/myc/cyto, pCR3.1, pSinRep5, DH26S, DHBB, pNMT1, pNMT41, pNMT81, which are available from Invitrogen, pCI which is available from Promega, pMbac, pPbac, pBK-RSV and pBK-CMV which are available from Strategene, pTRES which is available from Clontech, and their derivatives.

The polynucleotide (e.g., encoding JUNO and optionally the detectable marker) of some embodiments of the invention can be introduced into cells by any one of a variety of known methods within the art. Such methods can be found in any molecular biology handbook and include, for example, stable or transient transfection, lipofection, electroporation and infection with recombinant viral vectors.

According to a specific embodiment, the expression is in a stable manner, at least that of JUNO but optionally also of the detectable protein.

As expression of JUNO is critical for the assay, means may be taken to assure its expression such as by expressing a detectable protein on the same plasmid or nucleotide sequence (intervened by IRES, as mentioned above, as well as in the Examples section which follows).

Once somatic cells which express ectopically JUNO and optionally a detectable marker as explained herein are at hand, they can be contacted with the biological sample which comprises the sperm.

To achieve fusion, the biological sample needs to be capacitated.

As used herein “capacitation” is the set of physiological changes that the sperm undergoes during its transit through the female tract, after which it is capable of recognizing and fertilizing an oocyte [AUSTIN C R. Observations on the penetration of the sperm in the mammalian egg. Aust J Sci Res B. 1951 November; 4(4): 581-96. doi: 10.1071/bi9510581. PMID: 14895481. CHANG MC. Fertilizing capacity of spermatozoa deposited into the fallopian tubes. Nature. 1951 October 20; 168(4277): 697-8. doi: 10.1038/168697b0. PMID: 14882325. AUSTIN CR. The capacitation of the mammalian sperm. Nature. 1952 Aug. 23; 170(4321): 326. doi: 10.1038/170326a0. PMID: 129931]. In vitro capacitation involves the usage of a specific medium that mimics the conditions inside the female tract and allows the induction of this process in a petri dish [Visconti, P.E., D. Krapf, J. L. de la Vega-Beltrán, J. J. Acevedo, and A. Darszon. 2011. Ion channels, phosphorylation and mammalian sperm capacitation. Asian J. Androl. 13:395-405. www(dot)doi(dot)org/10.1038/aja.2010.69. Yanagimachi, R. 1994. Mammalian fertilization. In The Physiology of Reproduction. E. Knobil, and J. D. Neill, editors. Raven press, New York. 189-317).

Thus, the concentrated sperm is incubated in the capacitation medium under controlled conditions, such as temperature and pH. This allows the sperm to undergo the capacitation process. The sample is usually washed and concentrated to remove seminal plasma and other contaminants.

Capacitation medium is a specialized solution designed to mimic the conditions found in the female reproductive tract. It usually contains salts, sugars, and other nutrients to support sperm viability and motility, as well as bicarbonate ions to induce capacitation.

According to a specific embodiment, it comprises albumin and Ca⁺². See for example, Visconti P E Bailey J L Moore G D Pan D Olds-Clarke P Kopf G S (1995) Capacitation of mouse spermatozoa: I. Correlation between the capacitation state and protein tyrosine phosphorylation Development 121:1129-1137. www(dot)doi(dot)org/10.1242/dev.121.4.1129//Book

Yanagimachi R (1988) Chapter 1 Sperm-egg fusion In: Bronner Felix, editors. In Current Topics in Membranes and Transport. Academic Press. pp. 3-43.

Conditions for capacitation of human sperm are provided in the Examples section which follows (e.g., using mHTF medium). Human sperm typically requires Human Tubal Fluid (HTF) and mouse sperm requires mHTF (mouse HTF).

According to a specific embodiment, the amount of sperm and somatic cells in the reaction depends mainly on the level of expression of ectopic JUNO. Likewise, their selected ratio in the reaction might be affected.

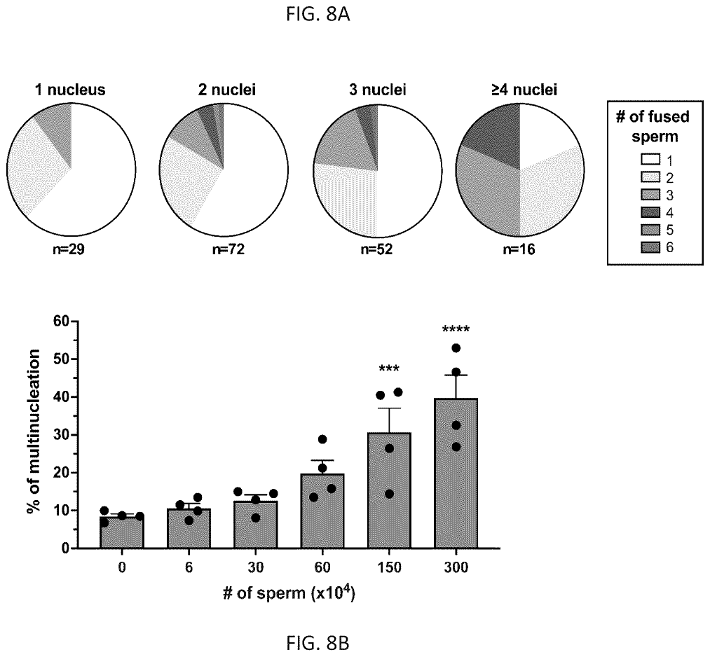

It has been shown in the Examples section that the fusion is dependent on the amount of sperm in the reaction, see FIG. 8.

Additional contacting conditions, which support fusion include, but are not limited to 37° C. and 5 % CO₂.

The mixture is incubated in said conditions for 1-6 hrs, e.g., 4 hours, after which multinucleation in the somatic cells is detected.

Detection typically relies on fluorescence or chromogenic detection. It will be appreciated that the somatic cell may not be transformed to express a detectable marker (at all) and that multinucleation will be detected by nuclear staining following the reaction. Such a nuclear staining may be DAPI staining or Hoechst.

Alternatively, the cells can express a marker to determine the base number of mononuclear cells (e.g., H2B:RFP).

As used herein “multinucleation” is interchangeable with “syncytia” or “syncytia formation” or also “hybrid cells” and “giant cells”.

Multinucleation can be detected by microscopy (manual counting or automated), by flow cytometry using DNA staining and therefore, detecting the amount of DNA per cell. Alternatively, cell-cell fusion can be detected by content-mixing (as described below) using microscopy or flow-cytometry. Moreover, there are content-mixing systems where a GFP or a Luciferace is split into two and expressed in two populations of cells. Therefore, after fusion, the whole protein is reconstituted and fluorescence or luminicense can be detected using a plate reader. This is called Dual Split Protein (Nakane and Matsuda Kurt Pfannkuche (ed.), Cell Fusion: Overviews and Methods, Methods in Molecular Biology, vol. 1313).

According to a specific embodiment, the level of multinucleation is calculated as % of multinucleation=[multinucleated cells (NuM)/total number of nuclei fluorescent cells (NuF)]×100.

According to a specific embodiment, when the level is above a predetermined threshold, it is indicative of competent sperm.

According to a specific embodiment, when the level is below a predetermined threshold, it is indicative of defective sperm.

The predetermined threshold is typically known from control studies, which assess the level achieved by a competent functional sperm sample as a positive control, whereby a decrease from this level by a certain value is indicative of a defective sperm.

The fusogenic potential assay described herein, can be harnessed to various research, breeding, clinical applications.

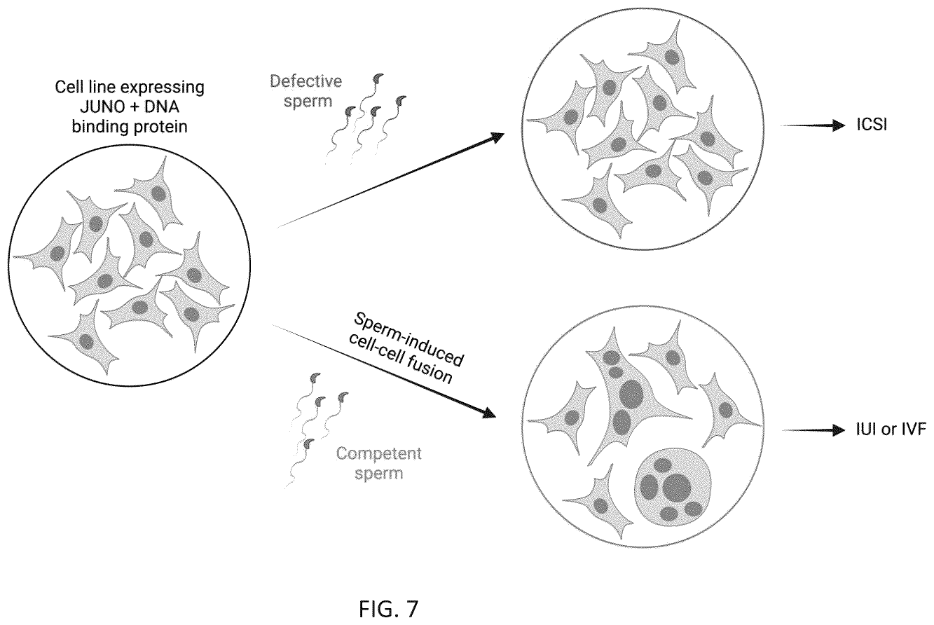

The assay could potentially predict the success chances of assisted reproductive therapies (ARTs) like intrauterine insemination (IUI) or conventional IVF (see illustration in FIG. 7), which require a fusion-competent sperm, and if it is necessary to proceed with a more complex technique such as intracytoplasmic sperm injection (ICSI). This is particularly important considering the routine use of the ICSI is under debate as it is associated with a slightly higher risk of adverse outcomes in the progeny. It is also envisaged that the assay will be able to resolve some unexplained cases of male infertility that result from loss of function of components of the fusion machinery. In this context, other proteins that are essential for gamete fusion, such as SPACA6, TMEM95, TMEM81, FIMP, SOF1, and DCST1/2 may be required together with IZUMO1 during sperm-induced cell-cell fusion. Hence, mutations or aberrations in any of them can be detected in the assay of some embodiments of the invention. The assay can be used to evaluate potential sperm donors and animal studs. Moreover, this assay can aid in screening for compounds that enhance fertilization (new fertility treatments) or that block gamete interactions (contraception), or to easily determine the effect of genetic variations of JUNO. The assay can also facilitate studies aimed at resolving standing enigmas of sperm-egg fusion.

Thus, according to an aspect of the invention there is provided a method of assisted fertilization in a subject in need thereof, the method comprising:
qualifying sperm for use in said assisted fertilization according to the method of determining fusogenic potential as described herein;
wherein when the sperm is found competent, directing the subject to intrauterine insemination (IUI) or in vitro fertilization (IVF) and wherein when the sperm is found defective directing said subject to intracytoplasmic sperm injection (ICSI).

According to another aspect of the invention there is provided a method of identifying an agent which affects fusogenic potential of sperm, the method comprising:
(a) contacting an aliquoted biological sample comprising sperm and somatic cells expressing ectopic JUNO under conditions which allow binding of said sperm to said JUNO in presence and in absence of an agent; and subsequently
(b) detecting multinucleation in said somatic cells in the presence and in the absence of said agent, wherein a change in level of said multinucleation is indicative of the effect of said agent on the fusogenic potential of the sperm.

This way it would be possible to identify fertility enhancers (when the fusogenic potential is enhanced in the presence of the agent) or contraceptives (when the fusogenic potential is inhibited in the presence of the agent).

The somatic cells which ectopically express JUNO and optionally the detectable marker can be included in a research or clinical kit with instructions for use along with sperm.

As used herein the term “about” refers to ±10 %.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

When reference is made to particular sequence listings, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion.

### Methods

### Animals

All animal studies were approved by the Committee on the Ethics of Animal Experiments of the Technion—Israel Institute of Technology. In this study, the present inventors used wild-type male mice from a FVB/129sv/CF1 mixed background were bred and housed in the Technion animal facility under specific pathogen-free conditions with ad libitum access to food and water. Male mice between 3 and 6 mo were used for the experiments.

### Cell Lines and DNA Transfection

In this study, the present inventors used BHK cells (Cat #CCL-10; ATCC, RRID: CVCL_1915) which were grown and maintained in DMEM containing 10% FBS. Cells were cultured at 37° C. in 5% CO2. Plasmids were transfected into cells using 2 μl jetPRIME (PolyPlus-transfection) per μg of DNA in 100 μl of reaction buffer for every ml of medium.

### Sperm Collection and Capacitation

Sperm were recovered by incising the cauda epididymis, obtained from adult male mice, in 300 μl of mHTF medium (Kito et al. 2004) supplemented with 4 mg/ml of BSA. The sperm were diluted in fresh medium to a concentration of 5×10⁶ cells/ml and incubated for 90 min at 37° C. and 5% CO₂ to induce capacitation.

### Sperm-to-BHK Fusion and Multinucleation

BHK cells were grown on 24-well glass bottom tissue-culture plates. 24 h after plating, cells were transfected with 0.25 μg pcDNA 3.1 -EGFP-MBD-nls plasmids and 0.5 μg of either pCI::H2B-RFP or pCI::JUNO::H2B-RFP. 24 h after transfection, 2×10⁶ capacitated wild-type sperm cells in mHTF were added to each well and co-incubated with the BHK cells for 4 hs at 37° C. and 5% CO₂. After one wash with PBS, the cells were fixed with 4% PFA in PBS and stained with 1 μg/ml DAPI. Micrographs were obtained using wide-field illumination using an ELYRA system S.1 microscope (Plan-Apochromat 20×NA 0.8; Zeiss). Multinucleation percentage was determined as the ratio between the number of nuclei in multinucleated cells (NuM) and the total number of nuclei fluorescent cells (NuF), as follows: % of multinucleation=(NuM/NuF)×100. 500 nuclei (NuF) were counted in each independent repetition (experimental point). In some cases, the number of sperm fused was determined by evaluating the transfer of EGFP-MBD-nls signal from the BHK cell to the sperm nuclei (number of fused sperm/500 BHK cells). In some experiments, sperm cells obtained from transgenic mice expressing IZUMO1-mCherry were employed to analyze IZUMO1 localization.

### Content Mixing Assay

BHK cells at 70% confluence in 35-mm plates were transfected with 1 μg pCI::H2B-RFP, pCI::GFPnes, pCI::JUNO::H2B-RFP or pCI::JUNO::GFPnes. 4 h after transfection, the cells were washed four times with DMEM with 10% serum, four times with PBS, and detached using Trypsin (Biological Industries). The cells were collected, resuspended in DMEM with 10% serum, and counted. Equal amount of H2B-RFP and GFPnes cells (1.25×10⁵ each) were mixed and seeded on glass-bottom plates (12-well black, glass-bottom #1.5H; Cellvis) and incubated at 37° C. and 5% CO₂. 18 h after mixing, 4×10⁶ capacitated wild-type sperm cells in mHTF were added to the BHK cells and co-incubated for 4 h after which they were washed with PBS, fixed with 4% PFA in PBS and stained with 1 μg/ml DAPI. Micrographs were obtained using wide-field illumination using an ELYRA system S.1 microscope (Plan-Apochromat 20×NA 0.8; Zeiss). The percentage of mixing was defined as the ratio between the nuclei in mixed cells (NuM) and the total number of nuclei in mixed cells and fluorescent cells in contact that did not fuse (NuC), as follows: % of mixing=(NuM/[NuM+NuC])×100. 1,000 nuclei (NuM+NuC) were counted in each independent repetition (experimental point).

### In Vitro Fertilization Assays

Ovulated oocytes were obtained from females previously treated with an i.p. injection of pregnant mare serum gonadotropin (5 IU; #HOR-272, Prospec), followed by an i.p. injection of human chorionic gonadotropin (5 IU, #CG 5; Sigma-Aldrich) 48 hr later. Cumulus-oocyte complexes (COCs) were collected from the ampullae of induced females 12-15 hr after human chorionic gonadotropin administration in mHTF medium. COCs were inseminated with 5×10³ capacitated sperm and co-incubated in capacitation media for 3 hr at 37° C. and 5% CO₂. Then, the oocytes were washed, stained with 10 μg/ml Hoechst 33342 (Sigma), and observed using wide-field illumination using an ELYRA system S.1 microscope (Plan-Apochromat 20×NA 0.8; Zeiss). Eggs were considered fertilized when at least one decondensing sperm nucleus or two pronuclei were observed in the egg cytoplasm. For fusion quantification, oocytes were denuded from the cumulus and the ZP by sequential treatment with 0.3 mg/ml hyaluronidase (H3506; Sigma-Aldrich) and acid Tyrode solution (pH 2.5). ZP-free eggs were inseminated with 10³ capacitated sperm and co-incubated for 1 hr. Then, the eggs were processed as above and the number of decondensing sperm nuclei per oocyte was scored.

### Evaluation of Acrosome Reaction

The extent of the acrosome reaction was evaluated by Coomassie brilliant blue staining. Briefly, the sperm cells were fixed in 4% paraformaldehyde in PBS for 15 min at room temperature, washed with 0.1 M ammonium acetate (pH 9) by centrifugation, mounted on slides, and air dried. Slides were successively immersed 5 min in water, 5 min in ice-cold methanol, 5 min in water, and 2 min in 0.22% Coomassie brilliant blue solution (50% methanol and 10% acetic acid). After washing with water, the samples were mounted and observed under a light microscope (X200). Sperm were scored as acrosome-intact when a bright blue labeling was observed in the dorsal region of the head or as acrosome-reacted when no staining was observed. For each condition, 1000 sperm were counted.

### Statistics and Data Analysis

Results are shown as means±SEM. For each experiment, at least three independent biological repetitions were performed. The significance of differences between the averages were analyzed using one-way ANOVA as described in the legends (GraphPad Prism 9, RRID: SCR_002798). Figures were prepared with Photoshop CS6 (Adobe, RRID: SCR_014199), Illustrator CS6 (Adobe, RRID: SCR_010279), BioRender(dot)com (RRID: SCR_018361), and FIJI (ImageJ 1.53c, RRID: SCR_002285).

### Example 1

### The System According to Some Embodiments of the Invention

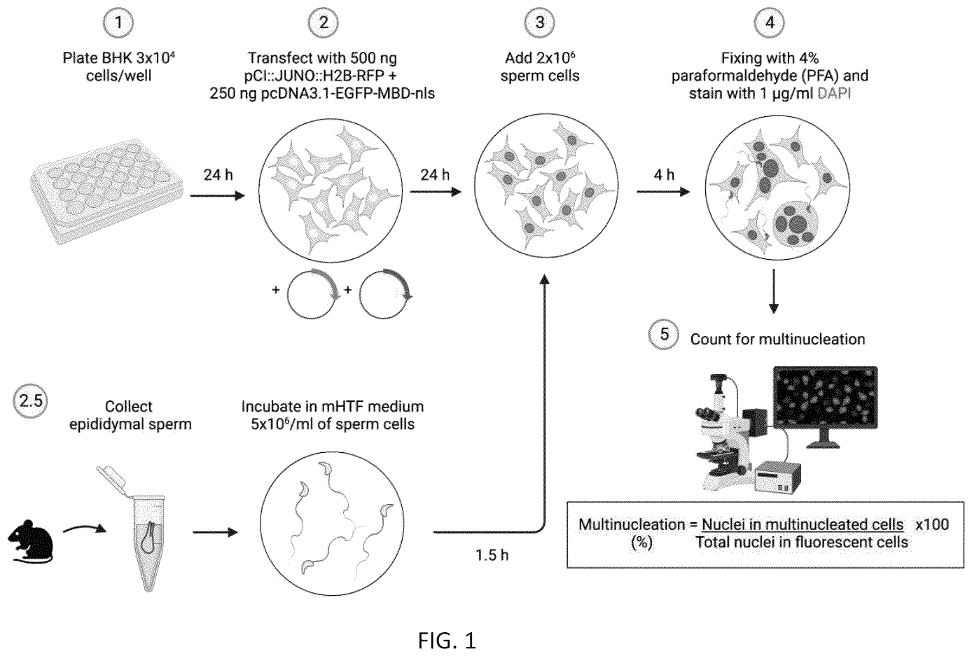

An embodiment of the system which measures sperm functionality is shown in FIG. 1. Sperm is co-incubated with Baby Hamster Kidney cells, previously transfected with pCI::JUNO::H2B-RFP and pcDNA 3.1-EGFP-MBD-nls plasmids. After 4 hours of incubation, the samples are fixed and the percentage of nuclei in multinucleated cells is quantified (% of multinucleation) (FIG. 1). In some cases, the number of sperm fused is determined by evaluating the transfer of EGFP-MBD-nls signal from the BHK cell to the sperm nuclei (number of fused sperm/500 BHK cells).

### Example 2

### Sperm Cell Fusion to Somatic Cells Expressing Exogenous JUNO Form Syncytia

Sperm can fuse to somatic cells (Rival et al. 2019; Mattioli et al. 2009; Bendich, Borenfreund, and Sternberg 1974). In the present experimental conditions, sperm cells only fuse to Baby Hamster Kidney cells after they are induced to express the sperm-receptor, JUNO (Brukman et al. 2023). This fusion was confirmed by detecting the transfer of the DNA binding GFP (GFP-MBD) from the BHK cells to the sperm heads. Surprisingly, it was found that sperm fusion to BHK induces the formation of multinucleated cells or syncytia (FIGS. 2A-C). The induction of multinucleation was dependent on the presence of JUNO (FIGS. 2B-C), however, this effect is not due to JUNO expression alone as only cells with sperm fused to them form syncytia (FIG. 2C). Therefore, the fusion of sperm is required for inducing multinucleation of BHK cells.

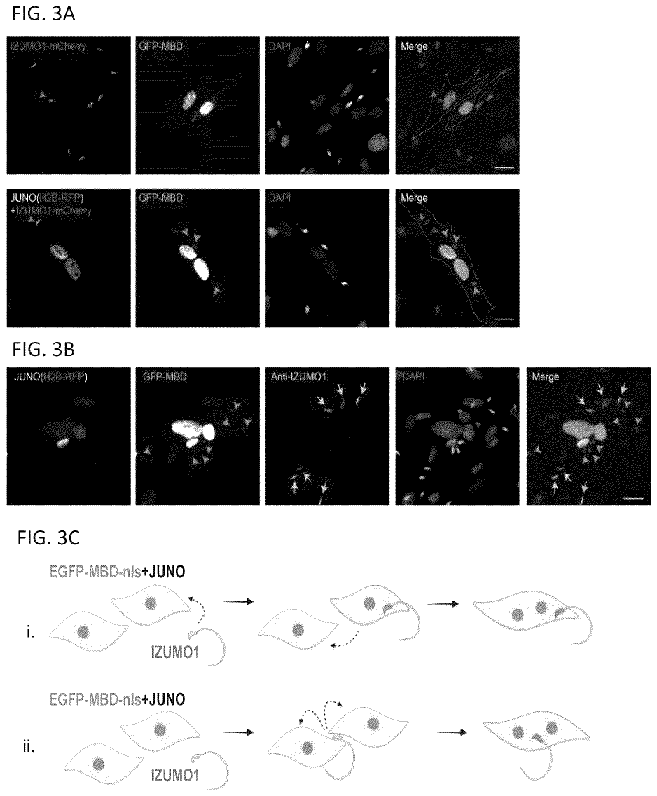

Previously, it was described that some somatic cells fuse one to each other after viral infection (Okada 1962; Kohn 1965). This process can require the synthesis of new proteins from the virus after the infection and therefore induce Fusion From Within (FFWI) while other viruses induce fusion independently of protein synthesis in a process called Fusion From Without (FFWO) (Bratt and Gallaher 1969). In a similar way, sperm could be inducing fusion in any of both ways and to distinguish between both alternatives the present inventors performed the experiment in the presence of cycloheximide to inhibit the synthesis of proteins. The results show no differences in the levels of multinucleation between the control and the cycloheximide treatments (FIGS. 2B and 2D). This confirms that protein synthesis is not required for the induction of multinucleation by sperm and suggests a mechanism of FFWO. The fact that IZUMO1, detected by immunostaining and by using a fluorescent reporter, is diffusing from the sperm head after its fusion to the BHK cell (FIGS. 3A-B) may be suggesting a role for the fusogenic machinery carried by the sperm during the FFWO process.

### Example 3

### Syncytia a Result of Juno Dependent Somatic Cells Fusion

Then the present inventors asked whether the sperm-induced multinucleation was a consequence of BHK-BHK fusion and if so, if it requires JUNO to be present in both fusing cells. For that a content mixing experiment was employed, where two populations of cells expressing different fluorescent markers are mixed and then exposed to sperm (FIG. 4A). The formation of multinucleated hybrid cells containing both markers was observed, confirming the fusion of BHK cells (FIG. 4B). This effect was not observed when one or none of the cell populations expressed JUNO (FIGS. 4B-C), indicating that sperm-induced fusion of BHK cells relies on the bilateral expression of JUNO. Consistent with the content-mixing assays, the presence of an inhibitor of the cell cycle (FdUdr, 5-fluoro-2′-deoxyuridine, (Valansi et al. 2017)) does not inhibit multinucleation (FIGS. 5A-B), ruling out that this effect is a consequence of failure in the division of the BHKs and confirming the occurrence of fusion between them.

### Example 4

### Multinucleation Can Be Used as a Readout of Sperm Fusogenic Potential

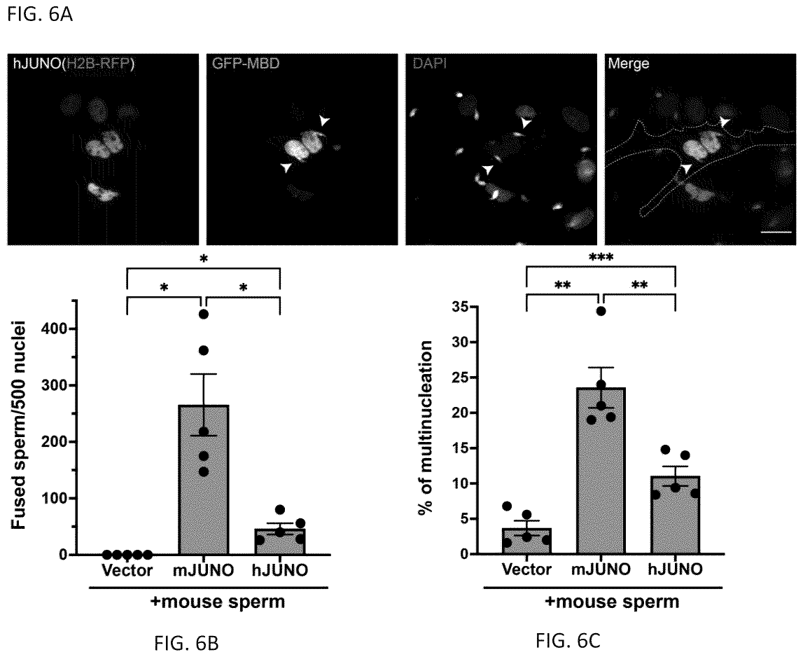

After confirming the ability of sperm to induce cell-cell fusion the present inventors decided to evaluate whether BHK multinucleation could be used as a readout of sperm fusogenic potential. For this purpose, the sperm was incubated in media lacking BSA or Ca²⁺ that do not support capacitation (i.e. a process by which sperm acquires its fusogenic activity) (Visconti et al. 1995). It was found that sperm cells incubated under these conditions failed to fuse to BHK cells, as well as to induce syncytia formation (FIGS. 5A-B). Moreover, a monoclonal antibody against IZUMO1, known to inhibit sperm-egg fusion (Inoue et al. 2013), managed to block both sperm-BHK and sperm-induced BHK-BHK fusions even when sperm cell were incubated in complete medium (FIGS. 5A-B). This antibody inhibition confirms that sperm-induced fusion depends on IZUMO1. Altogether, these results show that sperm cells without the ability to fertilize are not able to induce multinucleation, confirming that this assay is able to determine sperm fertilizing potential. Finally, in order to evaluate the species-specificity of the assay, the present inventors incubated the sperm with BHK cells expressing human JUNO. Under these conditions, lower levels of sperm-BHK fusion and induction of BHK multinucleation were found compared to cells expressing mouse JUNO, but still significant in comparison to the control without JUNO (FIGS. 6A-C). This confirms the requirement of a species-matching JUNO for the assay, however, puts in evidence a residual cross-interaction between mouse sperm and human JUNO.

### Example 5

### Assessing the Amount of Engineered Somatic Cells Needed to Analyze the Fusogenic Potential

The amount of sperm cells required to induce multinucleation was determined in 24-well plates. FIGS. 8A-B shows that the syncytia formation depends on the amount of sperm fused. In particular, FIG. 8A indicates that the number on sperm fused tends to be higher for bigger syncytia. FIG. 8B shows that 1.5-3×10⁶ sperm/well can produce statistically-significant increase in the percentage of multinucleation compared to the control without sperm.

### Example 6

### Cell-cell Fusion Correlates With the Fertilizing Ability of Sperm During in Vitro Fertilization Assays

FIG. 9 shows the results of in vitro fertilization assays made in parallel to multinucleation assessment for 10 different mice. Multinucleation levels are relative to the control without sperm as a function of the percentage of fertilized eggs when cumulus-oocytes complexes were used (Left panel), or as a function of the number of sperm fused per oocytes when ZP-free eggs were employed (Right panel). Each dot corresponds to a different mouse. The Pearson's coefficient ‘r’ and the significance are included in each panel. A positive and significant correlation was detected between the syncytia formation and the levels of fertilization, evaluated with complete and denuded oocytes. These results support the use of this assay as a predictor of sperm fertilizing ability.

### Example 7

Acrosome Reaction is Reduced in Non-Capacitated Sperm, but does not Correlate with SPERM-INDUCED Cell-cell Fusion in Capacitated Conditions.

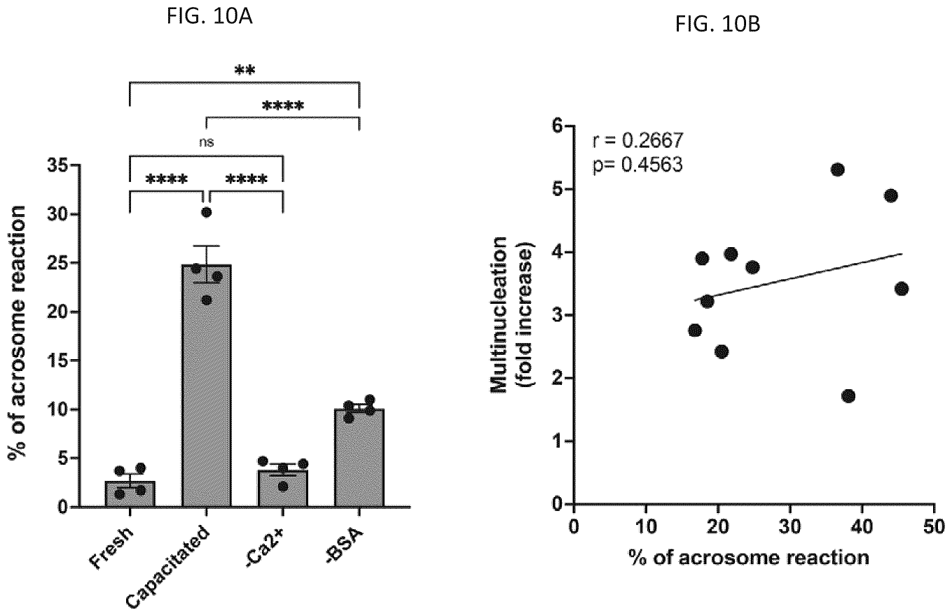

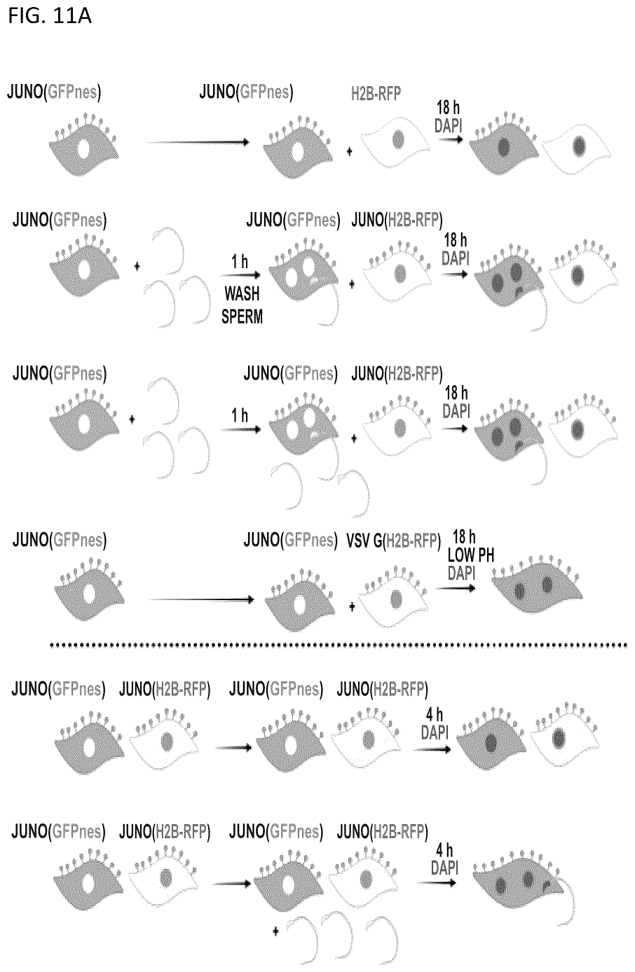

FIG. 10A shows the results of a control for FIGS. 5A-B). The percentage of acrosome reaction of fresh sperm, and sperm incubated in media lacking calcium (—Ca2+) or bovine serum albumin (-BSA) were significantly lower to those of capacitated sperm. FIG. 10B shows the multinucleation levels relative to the control without sperm (as shown in FIG. 9) as a function of the percentage of acrosome reaction after capacitation. Multinucleation did not correlate with the percentage of acrosome reaction of capacitated sperm, suggesting that the process relies not only on capacitation but on the overall sperm fertilizing potential.

### Example 8

### The Timing of Sperm Fusion to Somatic Cells is Critical for Content Mixing.

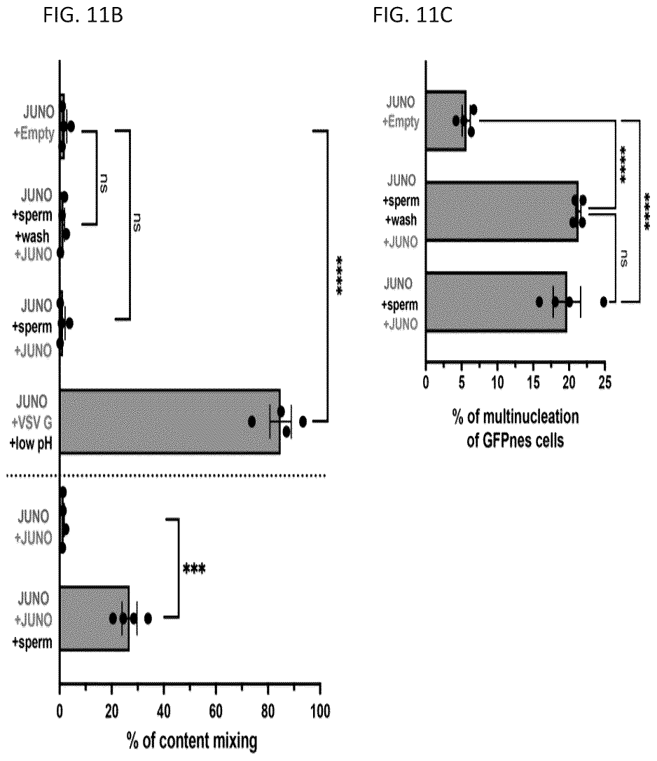

FIGS. 11A-C shows different timing setups for a color mixing experiment to determine the mechanisms of sperm-induced cell-cell fusion (FIG. 11A shows a scheme of the experimental design). In parallel, the regular mixing assay was performed FIGS. 11B-C When sperm were first allowed to fuse with one population of green cells before adding the second population of red cells, hybrid syncytia were not observed. Instead, exclusively GFP positive syncytia were detected. Only when a viral fusogen (VSV-G) was employed or when the two populations of cells were plated before the addition of the sperm, content mixing was induced. These results suggest that it is not possible to uncouple temporarily the sperm-BHK and BHK-BHK cell fusions, suggesting that sperm are fusing simultaneously to two cells using a sperm sandwich mechanism (FIG. 3Cii).

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the Applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

### REFERENCES

### Other References are Cited in the Document

Aitken, R. J., and R. A. Elton. 1986. “Quantitative Analysis of Sperm-Egg Interaction in the Zona-Free Hamster Egg Penetration Test.” International Journal of Andrology 9 (s6): 14-30.
Aitken, R. J., A. Ross, T. Hargreave, D. Richardson, and F. Best. 1984. “Analysis of Human Sperm Function Following Exposure to the Ionophore A23187. Comparison of Normospermic and Oligozoospermic Men.” Journal of Andrology 5 (5): 321-29.
Aydin, Halil, Azmiri Sultana, Sheng Li, Annoj Thavalingam, and Jeffrey E. Lee. 2016. “Molecular Architecture of the Human Sperm IZUMO1 and Egg JUNO Fertilization Complex.” Nature 534 (7608): 562-65.
Bendich, A., E. Borenfreund, and S. S. Sternberg. 1974. “Penetration of Somatic Mammalian Cells by Sperm.” Science 183 (4127): 857-59.
Bianchi, Enrica, Brendan Doe, David Goulding, and Gavin J. Wright. 2014. “Juno Is the Egg Izumo Receptor and Is Essential for Mammalian Fertilization.” Nature 508:483-87.
Bianchi, Enrica, and Gavin J. Wright. 2015. “Cross-Species Fertilization: The Hamster Egg Receptor, Juno, Binds the Human Sperm Ligand, Izumo1.” Philosophical Transactions of the Royal Society of London. Series B, Biological Sciences 370(1661): 20140101.
Bratt, Michael A., and William R. Gallaher. 1969. “PRELIMINARY ANALYSIS OF THE REQUIREMENTS FOR FUSION FROM WITHIN AND FUSION FROM WITHOUT BY NEWCASTLE DISEASE VIRUS*.” Proceedings of the National Academy of Sciences 64(2): 536-43.
Brukman, Nicolas G., Kohdai P. Nakajima, Clari Valansi, Kateryna Flyak, Xiaohui Li, Tetsuya Higashiyama, and Benjamin Podbilewicz. 2023. “A Novel Function for the Sperm Adhesion Protein IZUMO1 in Cell-Cell Fusion.” The Journal of Cell Biology 222 (2). www(dot)doi(dot)org/10.1083/jcb.202207147.
Clark, Sydney, and Rajesh K. Naz. 2013. “Presence and Incidence of Izumo Antibodies in Sera of Immunoinfertile Women and Men.” American Journal of Reproductive Immunology: AJRI: Official Journal of the American Society for the Immunology of Reproduction and the International Coordination Committee for Immunology of Reproduction 69 (3): 256-63.
Edwards, J., and D. T. Brown. 1986. “Sindbis Virus-Mediated Cell Fusion from without Is a Two-Step Event.” The Journal of General Virology 67 (Pt 2) (February): 377-80.
Enoiu, Simona Ioana, Marie Berg Nygaard, Mona Bungum, Søren Ziebe, Morten R. Petersen, and Kristian Almstrup. 2022. “Expression of Membrane Fusion Proteins in Spermatozoa and Total Fertilisation Failure during in Vitro Fertilisation.” Andrology, June. www(dot)doi(dot)org/10.1111/andr.13215.
Falke, D., A. Knoblich, and S. Müller. 1985. “Fusion from without Induced by Herpes Simplex Virus Type 1.” Intervirology 24 (4): 211-19.
Inoue, Naokazu, Masahito Ikawa, Ayako Isotani, and Masaru Okabe. 2005. “The Immunoglobulin Superfamily Protein Izumo Is Required for Sperm to Fuse with Eggs.” Nature 434 (7030): 234-38.
Inoue, N., D. Hamada, H. Kamikubo, K. Hirata, M. Kataoka, M. Yamamoto, M. Ikawa, M. Okabe, and Y. Hagihara. 2013. “Molecular Dissection of IZUMO1, a Sperm Protein Essential for Sperm-Egg Fusion.” Development 140:3221-29.
Kohn, a. 1965. “polykaryocytosis Induced by Newcastle Disease Virus in MONOLAYERS OF ANIMAL CELLS.” Virology 26 (June): 228-45.
Mattioli, M., A. Gloria, A. Mauro, L. Gioia, and B. Barboni. 2009. “Fusion as the Result of Sperm-Somatic Cell Interaction.” Reproduction 138 (4): 679-87.
Ohto, Umeharu, Hanako Ishida, Elena Krayukhina, Susumu Uchiyama, Naokazu Inoue, and Toshiyuki Shimizu. 2016. “Structure of IZUMO1-JUNO Reveals Sperm-oocyte Recognition during Mammalian Fertilization.” Nature 534 (7608): 566-69.
Okada, Y. 1962. “Analysis of Giant Polynuclear Cell Formation Caused by HVJ Virus from Ehrlich's Ascites Tumor Cells. I. Microscopic Observation of Giant Polynuclear Cell Formation.” Experimental Cell Research 26 (February): 98-107.
Palermo, G., H. Joris, P. Devroey, and A. C. Van Steirteghem. 1992. “Pregnancies after Intracytoplasmic Injection of Single Spermatozoon into an Oocyte.” The Lancet 340 (8810): 17-18.
Practice Committees of the American Society for Reproductive Medicine and the Society for Assisted Reproductive Technology. 2020. “Intracytoplasmic Sperm Injection (ICSI) for Non-Male Factor Indications: A Committee Opinion.” Fertility and Sterility 114 (2): 239-45.
Rival, Claudia M., Wenhao Xu, Laura S. Shankman, Sho Morioka, Sanja Arandjelovic, Chang Sup Lee, Karen M. Wheeler, et al. 2019. “Phosphatidylserine on Viable Sperm and Phagocytic Machinery in Oocytes Regulate Mammalian Fertilization.” Nature Communications 10 (1): 4456.
Satouh, Yuhkoh, Naokazu Inoue, Masahito Ikawa, and Masaru Okabe. 2012. “Visualization of the Moment of Mouse Sperm-Egg Fusion and Dynamic Localization of IZUMO1.” Journal of Cell Science 125 (Pt 21): 4985-90.
Steptoe, P. C., and R. G. Edwards. 1978. “Birth after the Reimplantation of a Human Embryo.” The Lancet 2 (8085): 366.
Tang, Jiajia, Giada Frascaroli, Xuan Zhou, Jan Knickmann, and Wolfram Brune. 2021. “Cell Fusion and Syncytium Formation in Betaherpesvirus Infection.” Viruses 13(10 ). www(dot)doi(dot)org/10.3390/v13101973.
Valansi, Clari, David Moi, Evgenia Leikina, Elena Matveev, Martín Graña, Leonid V. Chernomordik, Héctor Romero, Pablo S. Aguilar, and Benjamin Podbilewicz. 2017. “Arabidopsis HAP2/GCS1 Is a Gamete Fusion Protein Homologous to Somatic and Viral Fusogens.” The Journal of Cell Biology 216:571-81.
Visconti, Pablo E., Janice L. Bailey, Grace D. Moore, Dieyun Pan, Patricia Olds-Clarke, and Gregory S. Kopf. 1995. “Capacitation of Mouse Spermatozoa. I. Correlation between the Capacitation State and Protein Tyrosine Phosphorylation.” Development 121:1129-37.
World Health Organization. 2023. Infertility Prevalence Estimates, 1990-2021. Edited by World Health Organization. Geneva.
World Health Organization, HRP. 2021. “WHO Laboratory Manual for the Examination and Processing of Human Semen.” Sixth Edition. World Health Organization.
Yu, Mengru, Han Zhao, Tailai Chen, Ye Tian, Mei Li, Keliang Wu, Yuehong Bian, et al. 2018. “Mutational Analysis of IZUMO1R in Women with Fertilization Failure and Polyspermy after in Vitro Fertilization.” Journal of Assisted Reproduction and Genetics 35 (3): 539-44.

What is claimed is:

1. A method of determining fusogenic potential of sperm, the method comprising:
(a) contacting a biological sample comprising sperm with somatic cells expressing ectopic JUNO under conditions which allow binding of said sperm to said JUNO; and subsequently
(b) detecting multinucleation in said somatic cells, wherein a level of said multinucleation is indicative of the fusogenic potential of the sperm.

2. The method of claim 1, wherein said cells comprise epithelial cells.

3. The method of claim 1, wherein said cells comprise fibroblasts.

4. The method of claim 1, wherein said biological sample is a human sample.

5. The method of claim 1, wherein said cells are human cells.

6. The method of claim 1, wherein said cells are non-human cells.

7. The method of claim 1, wherein said cells express at least one detectable marker for said multinucleation.

8. The method of claim 1, wherein said cells comprise at least 2 subpopulation of cells each expressing a distinct detectable marker for said multinucleation.

9. The method of claim 7, wherein said detectable marker for said multinucleation is fluorescent or chromogenic.

10. The method of claim 7, wherein said detectable marker for said multinucleation is nuclear.

11. The method of claim 1, wherein said detecting comprise fluorescence or chromogenic detection.

12. The method of claim 11, wherein said detecting is by a nuclear stain or a nuclear localized reporter polypeptide.

13. The method of claim 1, wherein said conditions support capacitation of said sperm.

14. The method of claim 13, wherein said conditions comprise albumin and Ca⁺².

15. The method of claim 1, wherein when said level is above a predetermined threshold, it is indicative of competent sperm.

16. The method of claim 1, wherein when said level is below a predetermined threshold, it is indicative of defective sperm.

17. The method of claim 15, wherein said level is calculated as % of multinucleation=[multinucleated cells (NuM)/total number of nuclei fluorescent cells (NuF)]×100.

18. The method of claim 1, wherein said sperm is naïve.

19. A method of assisted fertilization in a subject in need thereof, the method comprising:
qualifying sperm for use in said assisted fertilization according to the method of claim 1;
wherein when said sperm is found competent, directing said subject to intrauterine insemination (IUI) or in vitro fertilization (IVF) and wherein when said sperm is found defective directing said subject to intracytoplasmic sperm injection (ICSI).

20. A method of identifying an agent which affects fusogenic potential of sperm, the method comprising:
(a) contacting an aliquoted biological sample comprising sperm and somatic cells expressing ectopic JUNO under conditions which allow binding of said sperm to said JUNO in presence and in absence of an agent; and subsequently
(b) detecting multinucleation in said somatic cells in the presence and in the absence of said agent, wherein a change in level of said multinucleation is indicative of the effect of said agent on the fusogenic potential of the sperm.
